(19) United States
(12) Patent Application Publication
(10) Pub. No.: US 2015/0297308 A1
(43) Pub. Date: Oct. 22, 2015

(54) ENTEROSCOPE MEASURING DEVICE

(71) Applicant: ENTEROGAUGE, LLC, Vernon Hills, IL (US)

(72) Inventors: Alex Padovano, Vernon Hills, IL (US); William Padovano, Vernon Hills, IL (US)

(73) Assignee: ENTEROGAUGE, LLC, Vernon Hills, IL (US)

(21) Appl. No.: 14/689,592

(22) Filed: Apr. 17, 2015

Related U.S. Application Data
(60) Provisional application No. 61/981,314, filed on Apr. 18, 2014.

(51) Int. Cl. A61B 19/00; A61B 1/04

(52) U.S. Cl. CPC A61B 19/46; A61B 1/041; A61B 2019/462

(57) ABSTRACT

The present invention is a measuring device for determining the insertion distance of an endoscope in a body lumen during an endoscopy. The measuring device generally may include a bracket member for attaching the measuring device to an endoscope, a spooling device for spooling thread wrapped around a thread disk attached to the spooling device, and a thread tension knob. Prior to an endoscopy, the biocompatible thread may be attached to the lumen interior, such as the small intestine wall. As the endoscope is removed from the lumen, the thread unwinds from the thread disk as the spooling device rotates during the spooling process. A counting device counts the rotations of the spooling device. The number of rotations may subsequently be translated to linear distance so that a location within the lumen may later be correlated to images taken during the endoscopy.

### CROSS-REFERENCE TO RELATED APPLICATIONS

This application claims the benefit of U.S. Provisional Application No. 61/981,314, filed Apr. 18, 2014.

### BACKGROUND OF INVENTION

The present invention relates generally to the medical field of gastroenterology. More particularly the present invention relates to the technical field of enteroscopy, or endoscopy of the small intestine, and distance measurements taken during such a procedure.

An enteroscopy is a nonsurgical procedure that is performed to examine a patient's digestive tract. Uses for an enteroscopy include evaluating and analyzing symptoms such as stomach pain, ulcers, gastritis, digestive tract bleeding, changes in bowel habits, or polyps or growths in the colon.

During an enteroscopy, an enteroscope is used by a doctor to record and analyze digestive tract images. An enteroscope typically includes a flexible tube with a light and camera attached to it that may be passed through the digestive tract such that images of the digestive tract may be captured and recorded. Those images may subsequently be analyzed to aid a physician with forming a strategy in addressing the above symptoms. An endoscope is a more general definition of an enteroscope, an enteroscope being a type of endoscope used for digestive tract procedures specifically. Other endoscopes are used for procedures involving other body systems.

Conventional methods for small bowel enteroscopy include the push-pull method, wireless capsule enteroscopy, and single or double bubble enteroscopy. Those conventional methods are typically expensive, inaccurate, and inefficient. The insertion distance of the enteroscope within the small bowel should be accurately measured so that a physician can identify problematic or otherwise noteworthy locations of the small intestine. When the aforementioned conventional methods are utilized, it is difficult to measure the small bowel insertion distance because there are few or no anatomical landmarks which can be used to measure the insertion distance within the small intestine.

For example, when using the capsule endoscopy method, which uses a capsule rather than an endoscope to obtain images of the small intestine, image capture may be efficient, but determining the location of those images on the small intestine may prove difficult. Attempting to measure insertion distance in order to obtain more precise and useful measurements using the conventional, known methods requires excessive amounts of time and too much estimation.

### SUMMARY OF INVENTION

The present invention is an enteroscope measuring device able to measure the depth of insertion of an enteroscope, or endoscope, using a spool mechanism. The measuring device may generally comprise a spooling device, a thread disk for holding a biocompatible thread (which may be a dissolvable suture thread) and spooling (spooling being used synonymously with “unwinding” herein) the thread into the small bowel, a tension knob for controlling the tension at which the suture thread is spooled (unwound), and a bracket member that joins the aforementioned components and may be able to affix the measuring device to an enteroscope or other endoscope device used during an enteroscopy or other endoscopy procedure. The measuring device may attach to the enteroscope or endoscope below the opening of the instrument's channel.

To use the present invention, a doctor or surgeon may attach a standard endoscopic clip to the free end of suture thread wrapped around the suture disk. For example, the endoscopic clip may receive a small loop tied at the end of the suture thread. Subsequently the endoscopic clip applicator, which is standard in endoscopic procedures, together with the strand of suture thread attached thereto may inserted into the instrument channel of the enteroscope or endoscope and advanced until the clip and suture thread loop emerge from the distal end of the instrument channel.

The doctor may then open the endoscopic clip with the suture attached to the endoscopic clip and anchor the endoscopic clip and suture thread loop of the absorbing suture thread to the walls of the small intestine. When the clip is detached from the applicator and the applicator catheter is withdrawn from the instrument channel the suture thread runs freely through the instrument channel as the spooling device rotates, thus connecting the spooling device to a fixed point in the intestine or other body lumen.

As the distal end of the enteroscope or endoscope moves relative to the fixed point in the small intestine, suture thread may be spooled from the suture disk while a counting device housed within the measuring device's bracket member may measure and record the radial revolutions of a pinwheel associated with the spooling device. Those revolutions may subsequently be converted to a distance, for example by the use of a microcontroller, and displayed on a digital display. The tension knob of the reel mechanism may be used to reduce any slack in the suture thread should the enteroscope or endoscope move toward the anchor clip.

After a procedure is completed, a physician may analyze the images taken by the camera housed in the enteroscope or endoscope (as known and understood in the prior art) while correlating those images with an insertion distance. That way, when abnormal or otherwise potentially problematic portions of the small bowel or other body lumen are observed, the physician may accurately identify the location of those abnormal or potentially problematic portions of the small bowel.

### DESCRIPTION OF THE SEVERAL VIEWS OF THE DRAWINGS

In the accompanying drawings, which form a part of the specification and are to be read in conjunction therewith in which like reference numerals are used to indicate like or similar parts in the various views:

FIG. 1 is a perspective view of an enteroscope measuring device according to the teachings of the present invention.

FIG. 2 is an exploded perspective view of the enteroscope measuring device of FIG. 1.

FIG. 3 is a side elevation view of the enteroscope measuring device of FIG. 1.

FIG. 4 is a front elevation view of the enteroscope measuring device of FIG. 1.

FIG. 5 is a rear elevation view of the enteroscope measuring device of FIG. 1.

FIG. 6 is a top plan view of the enteroscope measuring device of FIG. 1.

FIG. 7 is a bottom plan view of the enteroscope measuring device of FIG. 1.

FIG. 8 illustrates a perspective view of a bracket member of the enteroscope measuring device of FIG. 1.

FIG. 9 illustrates a side elevation view of the bracket member of FIG. 8.

FIG. 10 illustrates a front elevation view of the bracket member of FIG. 8.

FIG. 11 illustrates a rear elevation view of the bracket member of FIG. 8.

FIG. 12 illustrates a top plan view of the bracket member of FIG. 8.

FIG. 13 illustrates a bottom plan view of the bracket member of FIG. 8.

FIG. 14 illustrates a perspective view of a spooling device of the enteroscope measuring device of FIG. 1.

FIG. 15 illustrates a side elevation view of the spooling device of FIG. 14.

FIG. 16 illustrates a front elevation view of the spooling device of FIG. 14.

FIG. 17 illustrates a rear elevation view of the spooling device of FIG. 14.

### DETAILED DESCRIPTION OF THE INVENTION

The present invention is directed generally toward a device for measuring insertion distance during an endoscopy procedure. More particularly, the device is used for measuring enteroscope insertion distance into the small bowel during an enteroscopy procedure. It should be noted that while the device is described as being used during an enteroscopy procedure, the device may be used in a variety of endoscopy procedures, including but not limited to the following body parts: the respiratory tract, the ear, urinary tract, reproductive system, as well as other surgeries such as plastic surgery and endodontic surgery.

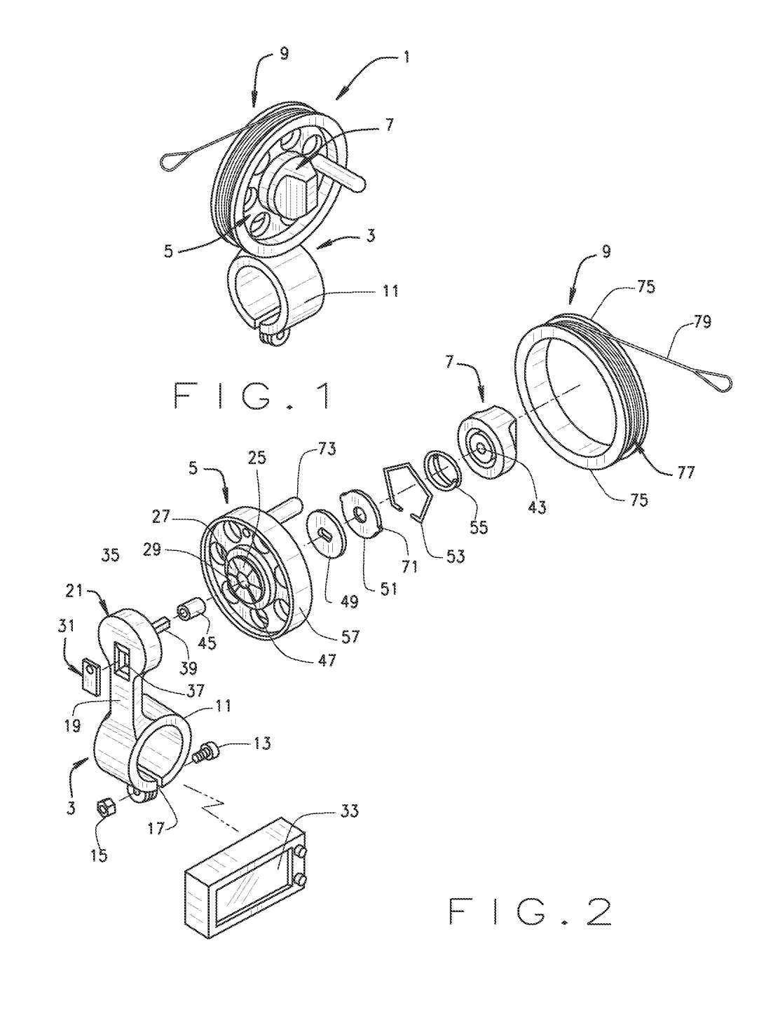

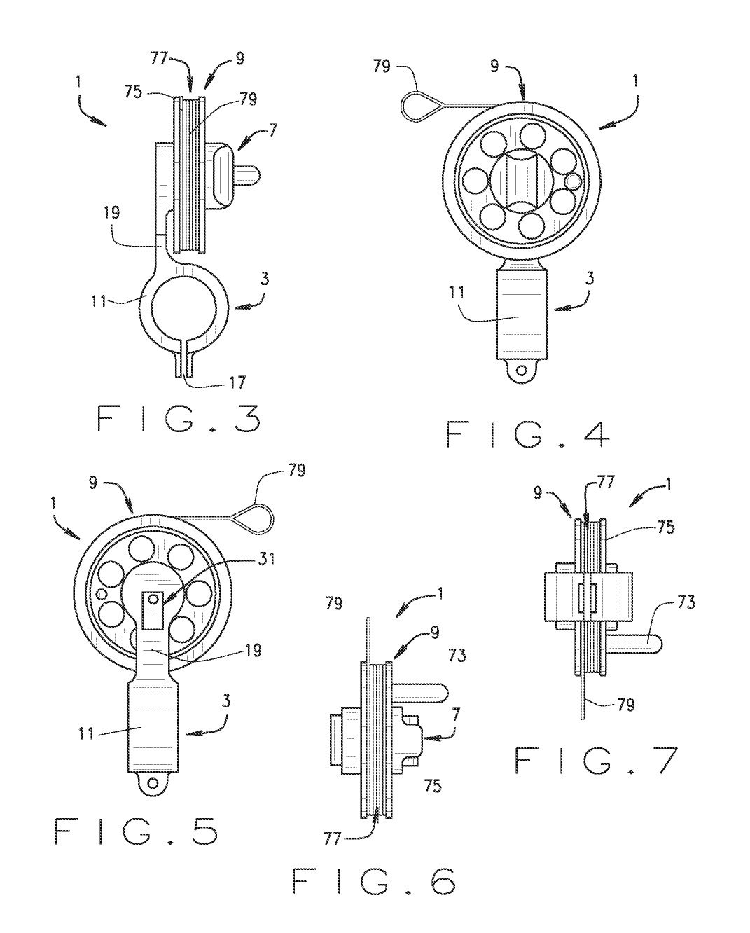

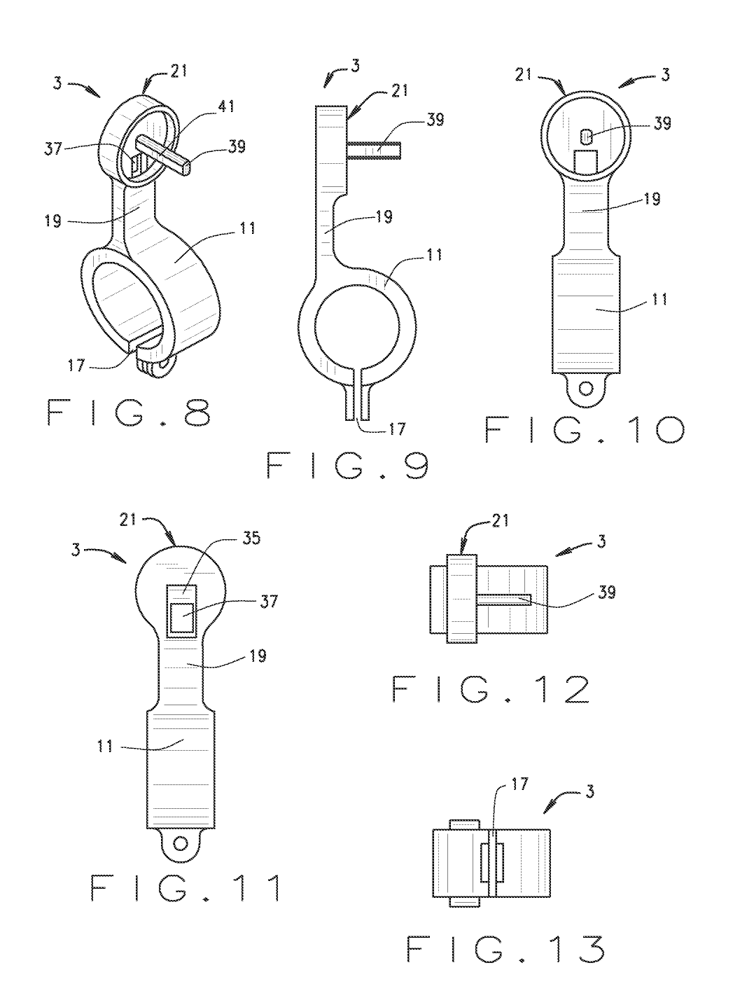

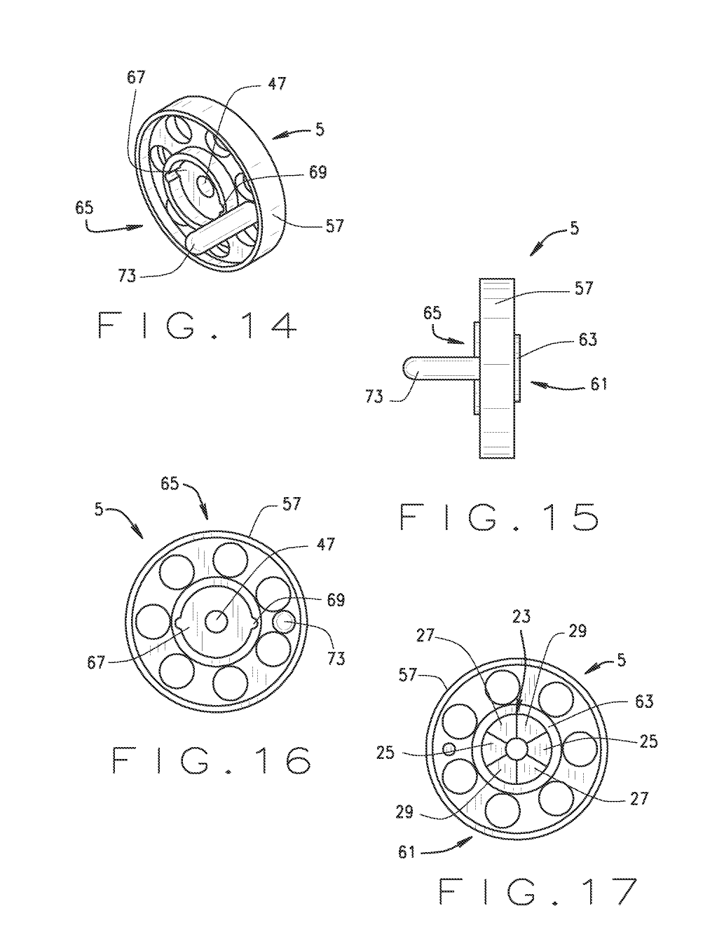

FIG. 1 illustrates a perspective view of an endoscope measuring device 1, and FIG. 2 illustrates an exploded perspective view of endoscope measuring device 1, referred to hereinafter as measuring device 1. Measuring device 1, as illustrated, may be used during an enteroscopy procedure. Measuring device 1 generally may comprise a bracket member 3, spooling device 5, tension knob 7, and suture disk 9. Bracket member 3, shown in FIGS. 1 and 2, and illustrated in detail in FIGS. 3-7, may be used to mount or otherwise secure measuring device 1 to an enteroscope handle (not illustrated) during an enteroscopy procedure.

A cylindrical mounting section 11 may be used to attach measuring device 1 to the enteroscope handle, as well understood in the art. Cylindrical mounting section 11 may have an inner diameter of 0.75″ to 1″, or any dimension sufficient to receive and engage an enteroscope handle, or other endoscope handle. Cylindrical mounting section 11 may be secured to an enteroscope handle using a bolt and nut mechanism such as that shown in FIG. 2 comprising bolt 13 and nut 15 as shown. Cylindrical mounting section 11 may include a gap 17 in its circumference such that cylindrical mounting section 11 may increase or decrease its inner diameter and thus be adapted for engagement with a variety of handle sizes for different types of endoscopes. Other means known or foreseeable in the art may be used to mount bracket member 3 to a handle or other foreseeable location on an enteroscope. Bracket member 3 also may include a neck member 19 that may operate to connect cylindrical mounting section 11 to an encoder housing 21 of bracket member 3. Neck member 19 may distance the axis of rotation of spooling device 5 from the enteroscope handle, such that spooling device 5 has sufficient clearance to rotate in the manner described in greater detail below when measuring device 1 is in use. In the illustrated embodiment, neck member 19 is approximately 1.5 inches long, though other sizes and lengths are envisioned.

Other means for attaching measuring device 1 to an enteroscope are foreseeable. In some embodiments attachment may not be necessary and measuring device 1 may be held by an operator manually or by a separate holding mechanism.

Encoder housing 21 may receive and engage encoder pinwheel 23 of spooling device 5. Encoder pinwheel 23 may be a circular arrangement of three colored segments 25, 27, 29. In the illustrated embodiment, colored segments 25, 27, 29 are generally triangular. In alternative embodiments, segments 25, 27, 29 may be alternative shapes. In yet other alternative embodiments, there may be greater or fewer segments. The three different and distinct colors of segments 25, 27, 29 preferably have unique absorptive properties, and thus may allow a counting device 31 to distinguish the distinct segments 25, 27, 29 and determine the angular position of spooling device 5 and pinwheel 23 thereof in the manner described below. In the preferred embodiment, the unique absorptive properties of segments 25, 27, 29 are in the infrared (IR) spectrum, but other spectrums are foreseeable so long as they are producible by an emitter and detectable by a sensor.

Counting device 31 may include an emitter of IR light (not illustrated), or other foreseeable type of light emission, that may reflect off of encoder pinwheel 23 and segments 25, 27, 29 associated therewith. Counting device 31 may also include a sensor (not illustrated) to measure the light absorption measured from segments 25, 27, 29 and an encoder (not illustrated) for encoding a resistance valve and/or encoder observed by the sensor. By sensing the changes in the absorptive properties in segments 25, 27, 29, the number of rotations of pinwheel 23 may be counted, preferably by a microcontroller (not illustrated) in communication with the sensor. Using the same microcontroller or other computational device, that number of rotations may be converted to a linear distance using a method well understood and long used by those skilled in the art. The distance measurements calculated may then be projected to an LCD screen 33 and subsequently correlated to images captured during the procedure.

Encoder housing 21 may also help to prevent ambient light from entering encoder housing 21 and thus interfering with any light absorption readings taken by counting device 31. As described in greater detail below, encoder pinwheel 23 and counting device 31 may communicate in order to measure the number and/or degree of rotations taken by spooling device 5 when measuring device 1 is in operation.

It should be noted that any measuring device or measurement method that is able to determine the insertion distance may suffice as an alternative to counting device 31. For example, in one alternative embodiment, an operator may measure the amount of thread that has been spooled into the small bowel by manually measuring the thread spooled during a procedure using known methods, and thus calculate an insertion distance. This and other foreseeable measurement means are illustrative only and are non-limiting examples.

Encoder housing 21 may also include a rectangular recess 35 for receiving and engaging counting device 31. In alternative embodiments, recess 35 may be a shape other than rectangular in order to accommodate a counting device 31 that is a shape other than rectangular. Counting device 31 may be secured within recess 35 in any number of foreseeable manners, but in the illustrated embodiment, counting device 31 may be engaged within recess 35 via a friction fit. Recess 35 may further include a window 37 that passes completely through encoder housing 21 so as to allow emitted IR light or other measurable light to reflect off of encoder pinwheel 23 and thus be detected by the components of counting device 31.

A cylindrical axle 39 may be attached to, or be integral with, encoder housing 21. Cylindrical axle 39 may be positioned and located such that it protrudes toward spooling device 5. Cylindrical axle 39 may be a rigid cylindrical axle including two flats 41. Other shapes for axle 39 are further foreseeable so long as threads on its distal portion match threads on the inside of tension knob 7. Cylindrical axle 39 may be ⅛″ to ¼″ in diameter and 1″ to 2″ in length, though other diameters and/or lengths are foreseeable so long as the size of axle 29 is sufficient to support spooling device 5 and may be received and engaged by tension knob 7. Tension knob 7 includes a threaded knob hole 43 for receiving and engaging cylindrical axle 39.

In the illustrated embodiment, axle 39 passes through a bearing, or bushing 45, in an aperture 47 in spooling device 5, keyed washers 49 and 51, retaining spring 53, and tension spring 55 before being received and engaged by threaded knob hole 43 of tension knob 7. Other means for selectively engaging bracket member 3 and tension knob 7 via spooling device 5 are further envisioned. Such alternative engagement means may include fewer washers such as washers 49, 51, fewer springs such as retaining spring 53 and tension spring 55, or may include more washers and/or springs.

As described above, spooling device 5 may be releasably engageable with bracket member 3. Spooling device 5 is illustrated by a perspective view in FIG. 14, and in other views showing greater detail in FIGS. 15-17. Spooling device 5 may include a spool wheel 57. Spool wheel 57 is preferably a cylindrically shaped wheel of approximately 2″ in outer diameter. Other diameter sizes are further contemplated herein. In the center of spool wheel 57, aperture 47 may receive bushing 45 of bracket member 3. An interior portion 61 of spool wheel 57 may include a raised surface 63 for supporting and engaging encoder pinwheel 23. In the illustrated embodiment, encoder pinwheel 23 is welded to raised surface 63, but in alternative embodiments, other means for attaching encoder pinwheel 23 to raised surface 63 are envisioned. In at least one alternative embodiment, encoder pinwheel 23 and raised surface 63 may be formed integrally.

An exterior portion 65 of spool wheel 57 may include a keyed recess 67 for receiving and housing keyed washers 49, 51, retaining spring 53, and tension spring 55. Keyed recess 67 may include two flats (not illustrated) keyed to flats 41 such that keyed recess 67 is keyed to cylindrical axle 39. Keyed washer 49 may also be keyed to cylindrical axle 39. As described above, any geometry may be keyed to allow cylindrical axle 39 and keyed recess 67 to be selectively engageable. Keyed recess 67 further may include semicircular receivers 69 for receiving and engaging semicircular protrusions 71 of keyed washer 51. The engagement of receivers 69 and protrusions 71 may help to secure keyed washer 51 and thus keyed washer 49 within keyed recess 67.

A handle 73 may further protrude from exterior portion 65 of spool wheel 57. When measuring device 1 is in use, handle 73 may be used to manually rotate spool wheel 57 in either a clockwise or counterclockwise direction so as to increase/decrease slack.

Spooling device 5, bushing 45, keyed washers 49, 51, retaining spring 53, and tension spring 55 may be received and engaged by threaded knob hole 43 as discussed above. Threaded knob hole 43 may be approximately ⅛″ in diameter, although other dimensions are further envisioned so long as those dimensions are able to receive cylindrical axle 39. When tension knob 7, spooling device 5 and bracket member 3 are engaged in the fashion described, tension spring 55 may push against keyed washers 49, 51 and thus spool wheel 57. The resultant force from tension spring 55 may increase rotational friction applied to spool wheel 57 and thus may prevent spool wheel 57 from rotationally spinning too rapidly. In the preferred embodiment, tension knob 7 is able to adjust the rotational friction exerted on spool wheel 57 simply by screwing or unscrewing tension knob 7 relative to cylindrical axle 39. This mechanism may allow an operator to fine tune the rotational resistance of measuring device 1 when in use.

Suture disk 9 may be a ring-shaped disc with raised edges 75 that together form a channel 77 for retaining and housing a biocompatible thread, such as suture thread 79. Any thread-like material is suitable, so long as it is unable to unwind without sticking to itself and is biocompatible. Dissolvable suture thread 79 is preferred because it is dissolvable and inexpensive.

Suture disk 9 may be made of rubber, plastic, or any other rigid material that is slightly elastic. Suture disk 9 may have an inner diameter only slightly larger than the outer diameter of spool wheel 57 such that when suture disk 9 is placed over spool wheel 57, suture disk 9 and spool wheel 57 may be engageable via a friction fit. Other means for releasably engaging suture disk 9 to spool wheel 57 are envisioned herein.

Channel 77 may be wrapped with several meters or more of suture thread 79 that may be used in the process described below to measure the depth of insertion of the enteroscope to which measuring device 1 is attached. Between procedures, suture disk 9 may be replaced by a new sterile suture disk that includes suture thread 79 that is also sterile.

Alternatively, suture disk 9 may not be necessary and thread 79 may be held by spooling device 5 or another foreseeable means. In any alternative embodiment not including spooling device 5, it is nevertheless preferable that thread 79 be biocompatible as well as sterile at the start of a procedure.

Prior to initiating an enteroscopy procedure, an endoscopic clip (not illustrated) may be attached to a loop, or simply a free end, of suture thread 79. Then, both the endoscopic clip and suture thread 79 may be advanced through the enteroscope until they both emerge together from the distal end thereof and are in the lumen of the intestine. The endoscopic clip, still attached to the loop or free end of suture thread 79, may then be attached to the wall of the intestine in a manner known and understood in the art, for example stitching. The endoscopic clip may then be detached from its applicator catheter, which may be withdrawn from the instrument channel (not illustrated). Thus, suture thread 79 may be anchored to a fixed point of the intestine at its free end or loop. At the same time, counting device 31 may be tared to zero such that its rotation counter is tared to zero.

When the distal end of the enteroscope moves relative to the fixed end of suture thread 79 to the intestine wall, suture 79 wrapped around channel 77 of suture disk 9 will be pulled therefrom and run through the instrument channel. Such movement of suture thread 79 may cause spool wheel 57, and thus pinwheel 23, and segments 25, 27, 29 of spooling device 5 to radially rotate. This rotation may be recorded by an optical encoder (not illustrated) of counting device 31. As described above, the counting device 31 may emit light at a particular wavelength and measure the light absorbed by segments 25, 27, 29 via a sensor. More segments of fewer segments may be used to increase or decrease measurement precision. The encoder may then record the number of rotations based on the absorption changes and transmit that data to the microcontroller circuitry. The microcontroller circuitry may translate the radial rotation data into a linear distance. Other means known or foreseeable for measuring the radial rotation distance and/or count are further envisioned. The calculated linear distance travelled by the suture thread 79 within the small intestine may be shown on LCD display 33. The linear distance information may (either in real time or after completion of a procedure) be correlated with images taken with a camera of the enteroscope during the enteroscopy procedure. Capturing photographic images during an enteroscopy procedure is well understood by those skilled in the art.

Upon completion of the procedure and/or measurement, suture thread 79 may be cut near the opening of the instrument channel and subsequently flushed into the patient's gastrointestinal tract with water or other biocompatible fluid. Suture disk 9 may subsequently be removed and discarded such that a new suture disk 9 may be attached prior to a subsequent procedure.

From the foregoing, it will be seen that this invention is one well adapted to attain all the ends and objects hereinabove set forth together with other advantages which are obvious and which are inherent to the structure. It will be understood that certain features and sub combinations are of utility and may be employed without reference to other features and sub combinations. This is contemplated by and is within the scope of the claims. Since many possible embodiments of the invention may be made without departing from the scope thereof, it is also to be understood that all matters herein set forth or shown in the accompanying drawings are to be interpreted as illustrative and not limiting.

The constructions described above and illustrated in the drawings are presented by way of example only and are not intended to limit the concepts and principles of the present invention. Thus, there has been shown and described several embodiments of a novel invention. As is evident from the foregoing description, certain aspects of the present invention are not limited by the particular details of the examples illustrated herein, and it is therefore contemplated that other modifications and applications, or equivalents thereof, will occur to those skilled in the art. The terms “having” and “including” and similar terms as used in the foregoing specification are used in the sense of “optional” or “may include” and not as “required”. Many changes, modifications, variations and other uses and applications of the present construction will, however, become apparent to those skilled in the art after considering the specification and the accompanying drawings. All such changes, modifications, variations and other uses and applications which do not depart from the spirit and scope of the invention are deemed to be covered by the invention which is limited only by the claims which follow.

What is claimed is:

1. A measuring device for measuring the insertion distance of an endoscope within a lumen, said device comprising:
a bracket member for selectively engaging said measuring device to an endoscope;
a spooling device engaged with said bracket member, said spooling device being rotatable relative to said bracket member; and
a counting device for determining the number of rotations of said spooling device during spooling.

2. The measuring device of claim 1, wherein the bracket member includes an encoder housing for containing the counting device.

3. The measuring device of claim 1, wherein the measuring device further includes a tension knob for adjusting a rotational resistance of the spooling device.

4. The measuring device of claim 1, wherein the spooling device includes a pinwheel, wherein the pinwheel is coded to be readable by the counting device.

5. The measuring device of claim 3, wherein the bracket member includes an axle for receiving and engaging the spooling device and the tension knob, and wherein the spooling device is rotatable about the axle.

6. The measuring device of claim 4, wherein the counting device includes a light emitter and a light absorption sensor.

7. The measuring device of claim 6, wherein the pinwheel includes a plurality of segments, each of the segments having different light absorption properties.

8. The measuring device of claim 7, wherein the counting device is able to detect via the light sensor the absorption properties of the plurality of segments.

9. The measuring device of claim 1, wherein the counting device is in communication with a microcontroller for calculating the linear distance of the thread within the lumen.

10. The measuring device of claim 1, wherein the measuring device includes a thread disk selectively engageable with the spooling device, said thread disk including a biocompatible thread for spooling.

11. A measuring device for measuring the insertion distance of an endoscope within a lumen, said measuring device comprising:
a spooling device, said spooling device being encoded such that its angular position may be measured, and said spooling device being rotatable;
a thread disk removably engageable with said spooling device, said thread disk being rotatable with said spooling device; and
wherein said counting device is able to determine rotational distance of said spooling device when said spooling device rotates.

12. The measuring device of claim 11, wherein the measuring device includes a bracket member selectively engageable with said spooling device, said bracket member including an encoder housing for containing the counting device.

13. The measuring device of claim 11, wherein the measuring device further includes a tension knob for adjusting a rotational resistance of the spooling device.

14. The measuring device of claim 11, wherein the spooling device includes a pinwheel, wherein the pinwheel is encoded to be readable by the counting device.

15. The measuring device of claim 13, wherein the bracket member includes an axle for receiving and engaging the spooling device and the tension knob, and wherein the spooling device is rotatable about the axle.

16. The measuring device of claim 14, wherein the counting device includes a light emitter and a light absorption sensor.

17. The measuring device of claim 16, wherein the pinwheel includes a plurality of segments, each of the segments having different light absorption properties.

18. The measuring device of claim 17, wherein the counting device is able to detect via the light sensor the absorption properties of the plurality of segments.

19. The measuring device of claim 11, wherein the counting device is in communication with a microcontroller for calculating the linear distance of the thread within the lumen.

20. The measuring device of claim 11, wherein the biocompatible thread is suture thread.
